(19) United States
(12) Patent Application Publication
(10) Pub. No.: US 2013/0035730 A1
(43) Pub. Date: Feb. 7, 2013

(54) IMPLANTABLE PULSE GENERATOR

(71) Applicants: John H. Erickson, Plano, TX (US); Galen L. Smith, Allen, TX (US); Robert E. Jones, McKinney, TX (US); Timothy S. Jones, Carrollton, TX (US)

(72) Inventors: John H. Erickson, Plano, TX (US); Galen L. Smith, Allen, TX (US); Robert E. Jones, McKinney, TX (US); Timothy S. Jones, Carrollton, TX (US)

(73) Assignee: ADVANCED NEUROMODULATION SYSTEMS, INC., Plano, TX (US)

(21) Appl. No.: 12/604,255

(22) Filed: Oct. 22, 2009

Related U.S. Application Data
(60) Provisional application No. 61/107,466, filed on Oct. 22, 2008.

(51) Int. Cl. A61N 1/375; H01R 43/00

(52) U.S. Cl. 607/2; 29/857

(57) ABSTRACT

In one embodiment, there is disclosed a lead locking mechanism for use in a header component of an implantable pulse generator. In certain embodiments, the lead locking mechanism comprises a first locking member positioned to engage a surface of at least two leads; a second locking member positioned to also engage a surface of at least two leads, and a coupling member coupling the first locking member to the second locking member.

### TECHNICAL FIELD

The present application is generally related to neurostimulation equipment used in the medical field, and in particular to header components of implantable pulse generators.

### BACKGROUND

Neurostimulation systems are devices that generate electrical pulses and deliver the pulses to nerve tissue to treat a variety of disorders. Spinal cord stimulation (SCS) is an example of neurostimulation in which electrical pulses are delivered to nerve tissue in the spine for the purpose of chronic pain control. Other examples include deep brain stimulation, cortical stimulation, cochlear nerve stimulation, peripheral nerve stimulation, vagal nerve stimulation, sacral nerve stimulation, etc. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

Neurostimulation systems generally include a pulse generator and one or more leads. The pulse generator is typically implemented using a metallic housing that encloses circuitry for generating the electrical pulses, control circuitry, communication circuitry, a rechargeable battery, etc. The pulse generation circuitry is electrically coupled to one or more stimulation leads through electrical connections provided in a “header” of the pulse generator. Typically, conductors in the leads carry the electrical pulses from electrodes which are inserted into the header to a longitudinal series of stimulation electrodes implanted at the tissue site.

Pulse generators are preferably small to limit patient trauma and discomfort. Similarly, there is a preference to shorten the implant procedure and to simplify the implant process.

### SUMMARY

In one embodiment, there is disclosed a lead locking mechanism for use in a header component of an implantable pulse generator. In certain embodiments, the lead locking mechanism comprises a first locking member positioned to engage a non-electrical conducting surface of at least two leads; a second locking member positioned to also engage a non-electrical conducting surface of at least two leads, and a coupling member coupling the first locking member to the second locking member, thereby locking the leads in place.

In another embodiment, there is disclosed a method of assembling an implantable pulse generator which includes: providing pulse generating circuitry within a housing; providing feed-through wires to permit electrical access to the pulse generating circuitry within the housing; coupling a first portion of the feed-through wires to a first plurality of longitudinally arranged electrical conductors; coupling a second portion of the feed-through wires to a second plurality of longitudinally arranged electrical conductors; inserting conducting electrodes of a first lead into the first plurality of longitudinally arranged annular electrical conductors, inserting conducting electrodes of a first lead into the first plurality of longitudinally arranged annular electrical conductors, moving a first locking member towards a second locking member such that the first locking member and the second locking member engages both the first lead and the second lead to secure the position of the first and second leads relative to the electrical conductors.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 is an isometric view of a pulse generation system incorporating certain aspects of the present invention.

FIG. 2a is an exploded isometric view of a header used in the pulse generation system of FIG. 1.

FIG. 2b is an isometric view of a portion of the header of FIG. 2.

FIG. 3a is a partial isometric view of the lead receiving assembly which may be used in the header of FIG. 2a.

FIG. 3b is a partial exploded.isometric view of the lead locking mechanism which may be used with the lead receiving assembly of FIG. 3a.

FIG. 4 is an isometric view of one embodiment of a lead locking mechanism.

FIG. 5 is an isometric view of an alternative embodiment of a lead locking mechanism.

FIG. 6 is a section view of the header of FIG. 2 illustrating details of the lead locking mechanism.

### DETAILED DESCRIPTION

Some representative embodiments are directed to a header design for a neurostimulation system. In one design, the header design might incorporate a lead locking mechanism for use in a header component of an implantable pulse generator. In certain embodiments, the lead locking mechanism comprises a first locking member positioned to engage a non-electrical conducting surface of at least two leads; a second locking member positioned to also engage a non-electrical conducting surface of at least two leads, and a coupling member coupling the first locking member to the second locking member to secure the position of the leads.

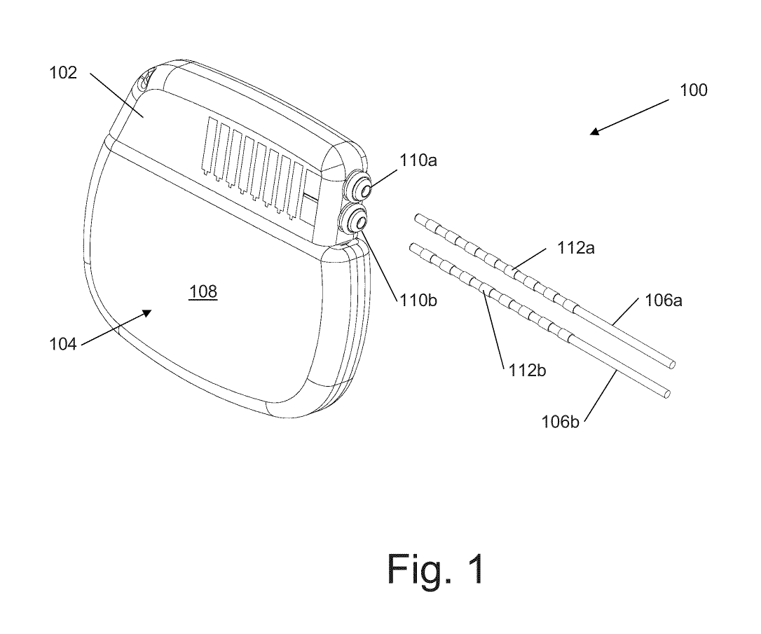

Turning now to FIG. 1, there is presented an isometric view of one embodiment of an implantable pulse generator (IPG) system 100 which may incorporate one or more embodiments of the present invention. The IPG system 100 includes a header 102, a stimulation source, such as an embodiment of an implantable pulse generator (IPG) 104, and at least one stimulation lead. In this illustrative embodiment, the proximal ends of the two stimulation leads 106a and 106b are shown.

As is well known in the art, the IPG 104 is capable of being implanted within a body (not shown) that is to receive electrical stimulation from implantable pulse generator circuitry. In certain embodiments, the IPG 104 may comprise a metallic housing 108 that encloses the pulse generating circuitry, control circuitry, communication circuitry, battery, etc. of the device. An example of pulse generating circuitry is described in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. An embodiment of a microprocessor and associated charge control circuitry for an implantable pulse generator which may be used in certain embodiments is described in U.S. Patent Publication No. 20060259098, entitled “SYSTEMS AND METHODS FOR USE IN PULSE GENERATION,” which is incorporated herein by reference. Examples of circuitry for recharging a rechargeable battery of an implantable pulse generator using inductive coupling with an external charging device are described in U.S. patent Ser. No. 11/109,114, entitled “IMPLANTABLE DEVICE AND SYSTEM FOR WIRELESS COMMUNICATION,” which is incorporated herein by reference. An example of a commercially available implantable pulse generator that may be adapted to include the header 102 according to some representative embodiments is the EON® implantable pulse generator available from Advanced Neuromodulation Systems, Inc.

In this illustrative example, the header 102 electrically couples the stimulation leads 106a and 106b to the circuitry of the implantable pulse generator 104 when the leads 106a and 106b are inserted into strain relief ports 110a-110b and properly positioned within the header 102. In certain embodiments, the two strain relief ports 110a and 110b are adapted to receive the proximal ends of stimulation leads 106a and 106b in an above-below manner. Other embodiments may be configured to receive the stimulation leads in a side-by-side manner or even in an array-like manner if several stimulation leads are coupled to the header. Although only two stimulation leads are shown in one embodiment, any number of stimulation leads may be secured in header 102 using a single locking mechanism disclosed herein according to alternative embodiments.

At the proximal end portion of the stimulation leads 106a and 106b, there may be a plurality of connector electrodes 112a and 112b which are coupled to conductors (not shown) running longitudinally within the leads 106a and 106b. For purposes of illustration only, the leads 106a and 106b are shown with eight connector electrodes. As will be appreciated by those skilled in the art, any number of connector electrodes may be utilized as desired within the leads 106a and 106b. In this illustrative embodiment, the pluralities of connector electrodes 112a and 112b are shown as band or ring electrodes. In certain embodiments, the connector electrodes may be formed of biocompatible, conductive materials which do not develop a significant amount of oxide films, such as platinum and platinum-iridium, or other conductive materials, metals or alloys known to those skilled in the art.

As will be explained in detail below, when the IPG system 100 is assembled, the connector electrodes 112a-112b are electrically coupled to feed-through wires (not shown) positioned within the header 102. The feed-through wires, therefore, connect the connector electrodes 112a-112b to pulse generating circuitry (not shown) within the pulse generator 104. The connector electrodes are themselves electrically coupled to stimulation electrodes (not shown) positioned at distal ends (not shown) of the leads 106a and 106b via the longitudinal conductors.

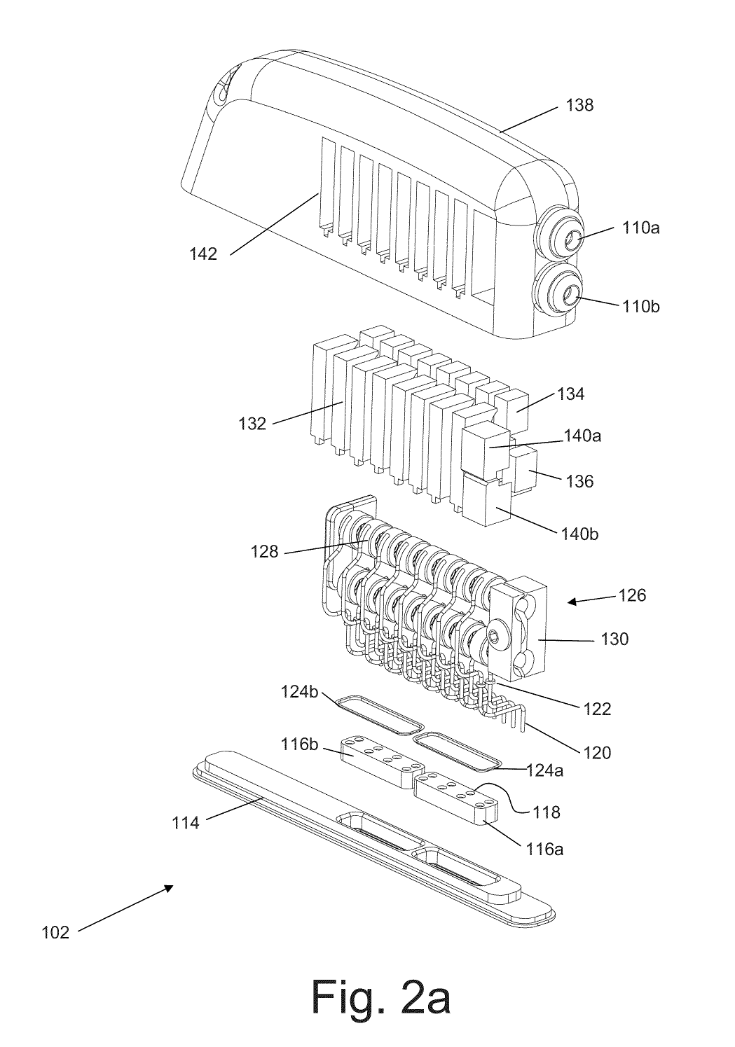

Details of the header 102 will be discussed with reference to FIG. 2a which is an exploded view illustrating various components of one embodiment of the header. As can be seen in FIG. 2a, in the illustrated embodiment, there may be a feed through base 114, which in certain embodiments may be glued or mechanically attached to the IPG 104 (FIG. 1). In certain embodiments, the feed through base 114 may shaped to house a plurality of supports 116a and 116b arranged longitudinally within the feed through base 114. In certain embodiments, the supports 116a and 116b may be made of ceramic and may have a plurality of cylindrical passages or bores 118 which allow a plurality of feed-through wires 120 to extend through. (For purposes of this application, the term “bore” is defined as a cylindrical shaped hole.)

On the proximal face of the supports 116a-116b, the diameter of the bores may be enlarged to house a plurality of wire seals 122 which may be coupled to the feed-through wires 120. In certain embodiments, there may also be base seals 124a and 124b which fit between the feed-through base 114 and the supports 116a and 116b to form seals between the supports and the feed through base. (For ease of explanation in this specification, the direction towards the IPG 104 and away from the header 102 will be defined or referred to as the distal direction and the opposing direction will be defined as the proximal direction. Similarly, in use, the direction away from the user will be defined to be the distal direction and the direction towards the user will be defined to be the proximal direction.)

As will be explained in detail below, the feed through wires 120 are coupled to a lead receiving assembly 126, which receives and secures the proximal end of the leads 106a and 106b (FIG. 1) to the header 102. In certain embodiments, the lead receiving assembly 126 comprises a plurality of annular electrode connectors 128 and a lead locking mechanism 130. The feed through wires 120 are electrically coupled (e.g., welded) to annular electrical connectors 128. When the IPG system 100 is assembled, each connector electrode in the plurality of connector electrodes 112a-112b (FIG. 1) contacts one of the annular electrical connectors 128 and, thereby, is electrically coupled to the pulse generating circuitry through the feed through wires 120.

Three sets of positioning blocks 132, 134 and 136 secure the annular connectors 128 in a fixed arrangement to correspond to the connector electrodes 112a-112b. In tum, the positioning blocks 132, 134, and 136 are positioned and held within the structure of the header housing 138. Thus, the header housing 138 in combination with the positioning blocks 132, 134 and 136 hold the annular connectors in a fixed arrangement that corresponds to the arrangement of connector electrodes on a stimulation lead. In certain embodiments, the header housing 138 also holds a pair of septum seal members 140a and 140b.

In certain embodiments, the sets of positioning blocks 132, 134, and 136 and the septum seal members 140a and 140b may be formed from a compliant material, such as silicone rubber or soft polyurethane. The positioning blocks are shaped to frictionally fit within corresponding slots or holding compartments 142 formed within the header housing 138. In certain embodiments, the header housing 138 may also be formed of a compliant material, such as silicone rubber. In yet other embodiments, the header housing 138 may be formed from a bio-compatible rigid material, such as Bionate® polycarbonate urethane.

As previously discussed, the strain relief ports 110a-110b may be formed within the header housing 138. The strain relief ports 110a-110b are sized to accept stimulation leads 106a-106b (FIG. 1). In certain embodiments, additional annular seals (not shown) may be provided within the header housing 138 proximal to the strain relief ports 110a and 110b to provide for additional sealing between the header housing 138 and the leads 106a and 106b (FIG. 1).

FIG. 2b is an isometric view of a partially assembled header 102 with the header housing 138 and lead locking mechanism 130 removed for clarity. As illustrated, the feed through wires 120 extend through the supports 116a-116b (support 116b is not visible in FIG. 2b) which are positioned within the feed through base 114. A portion of the positioning blocks, 132, 134, and 136 are illustrated in position which, in turn, maintains the position of the electrode connectors 128.

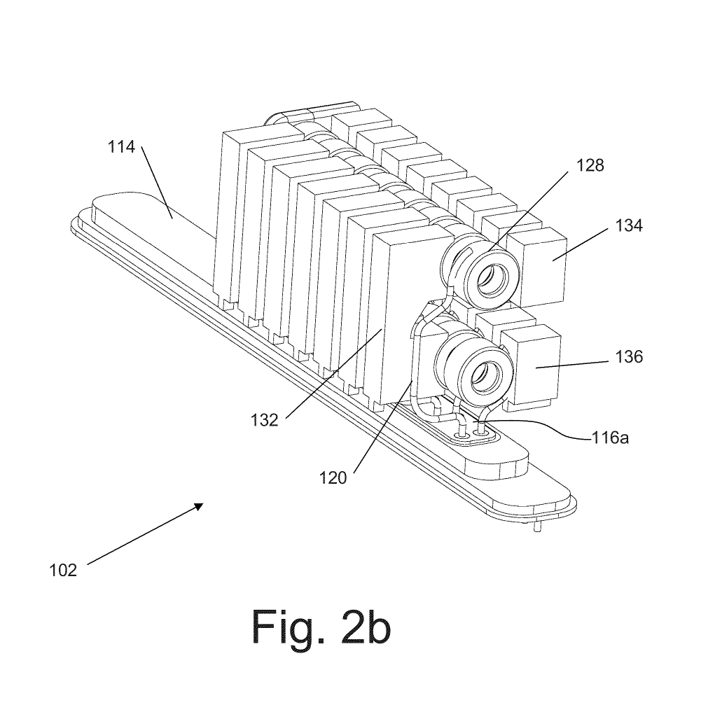

FIG. 3a is an isometric view of the lead receiving assembly 126. In this embodiment, the lead receiving assembly 126 comprises two rows or stacks 128a and 128b of the annular electrical connectors 128. Each stack 128a-128b of electrical connectors 128a and 128b are aligned axially along a longitudinal axis to correspond with the spacing of the connector electrodes 112a or 112b which are longitudinally positioned along the corresponding lead 106a-106b (FIG. 1). The stacks 128a-128b are positioned vertically to align with the respective strain relief port 110a-110b of the header housing 138 (FIG. 2a).

Typically, the annular electrical connectors 128 are fabricated using an outer conductive annular or ring-like structure. Within the ring-like structure, one or more conductive members (such as spring members) are held to engage a respective electrode (e.g. electrodes 112a or 112b) of the stimulation lead. An example of a known connector uses a canted spring held within a conductive ring or circular support member. Such connectors are commercially available from Bal Seal, Inc. of Foothill Ranch, Calif. Another example of a known connector or spring member that uses a conductive disk having arcuate connector tabs (or springs) held within a conductive ring as described in U.S. Patent Publication No. 20050107859, entitled “SYSTEM AND METHOD OF ESTABLISHING AN ELECTRICAL CONNECTION BETWEEN AN IMPLANTED LEAD AND AN ELECTRICAL CONTACT,” which is incorporated herein by reference. It shall be appreciated that other types of electrical connectors could be employed such as “block electrical connectors” which are known in the art. Also, different types of electrical connectors could be employed within the same header in any suitable configuration. Both the feed-through wires 120 and the corresponding annular ring of the electrical connectors 128 may be made from platinum, a platinum-iridium alloy, or other metals that produce non-oxide or low oxide film on their surfaces. In certain embodiments, traditional conductor materials, such as copper, nickel or gold plated alloys may also be used.

In certain embodiments, there may be at least one lead stop member 144 positioned at the end of the stacks 128a and 128b to stop the progress of the lead (not shown) when the lead is inserted through the stacks of electrical connectors. The lead stop member 144 may be held in position by a corresponding cavity formed within the header housing 138 (FIG. 2). Once the lead has been properly inserted into the appropriate electrode stack, the lead locking mechanism 130 may be used to secure the lead in place.

FIG. 3b is a partial exploded isometric view of the lead locking mechanism 130 and an isometric view of the rest of the lead receiving assembly 126. In the illustrative embodiment, the lead locking mechanism 130 comprises a coupling member, such as cap screw 146, a first locking member 148, a second locking member 150, and a compliant member 152.

The cap screw 146, the first locking member 148, and the second locking member 150 may be made from a relatively hard material, such as stainless steel or platinum. In certain embodiments, the first locking member 148 may be a generally rectangular shaped member having a center bore 154 running transversely through the member. The center bore 154 has an interior diameter sized to allow the insertion and passage of the cap screw 146. In certain embodiments, the second locking member 150 may be a generally rectangular shaped member having a center bore 156 running transversely through the member. In certain embodiments, the center bore 156 may have a threaded portion (not shown) to receive corresponding helical threads 157 on the shaft of the cap screw 146. As will be explained below, both the first locking member 148 and the second locking member 150 have surfaces designed to engage the leads when the lead locking mechanism 130 is fully assembled.

In certain embodiments, the compliant member 152 may be an O-ring. The compliant member may be made from any resilient bio-compatible material. In certain embodiments, the compliant member 152 maintains a distance or spacing between the first locking member 148 and the second locking member 150 so that a lead (not shown) may be easily inserted between the locking members when the locking mechanism 130 is in an un-locked position. As the first locking member 148 is moved closer to the second locking member 150, the compliant member 152 compresses in response to the compressive forces which will allow the first locking member and the second locking member to hold or secure the lead. The compliant member 152 may also assist in the separation of the two connecting block components and will make unscrewing easier if revisions are required later. Although the compliant member 152 is shown as an O-ring made from compliant material, the compliant member might also be a metal spring that would apply a force to separate the two locking members.

As the cap screw 146 is rotated clockwise, the threads 157 on the screw shaft engage the threads defined within the center bore 156 of the second locking member 150 which moves the cap screw 146 towards the second locking member. Because the cap screw 146 is coupled to the first locking member 148, clockwise movement of the cap screw 146 causes the first locking member to be pushed closer to the second locking member 150, thereby engaging the leads 106a-106b when the IPG system 100 (FIG. 1) is assembled.

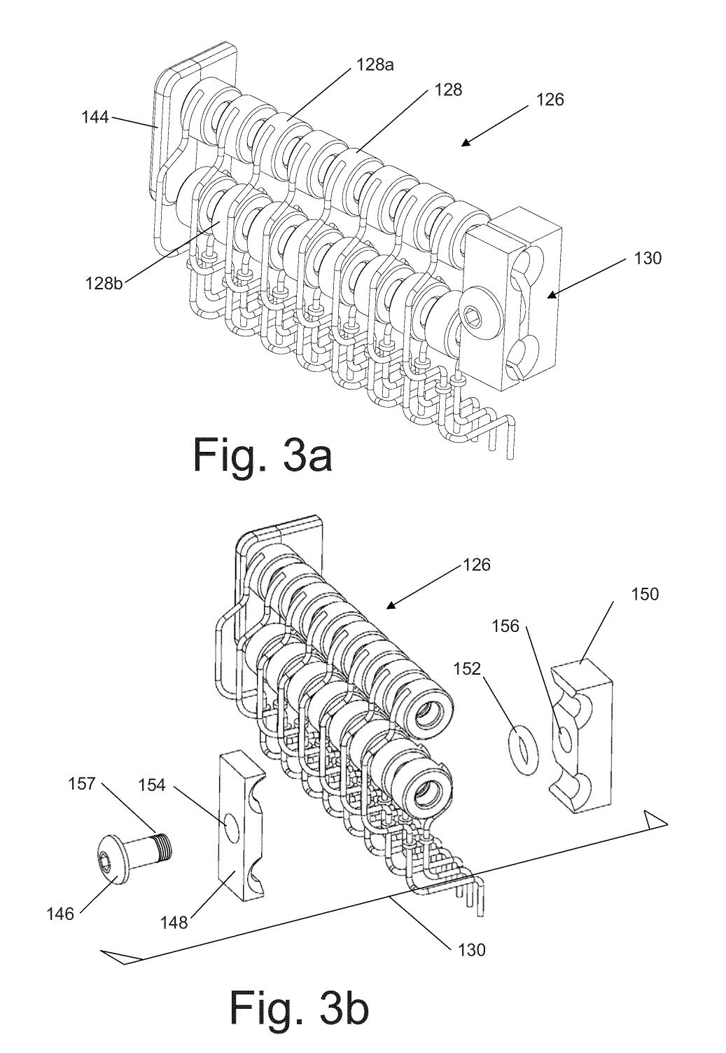

The locking members 148 and 150 may have various surfaces and features which allow the locking members to better engage the surface of the respective lead. FIG. 4 is an illustrative example of the locking mechanism 130. FIG. 4 shows the coupling member or cap screw 146 coupling the first locking member 148 to the second locking member 150. In this example, the compliant member 152 is positioned between the first locking member 148 and the second locking member 150. Formed within each engaging face of the respective locking members 148 and 150 are one or more circular indents 158a and 158b. The circular indents 158a and 158b are sized to engage the round surface of a lead. In certain embodiments, the circular indents 158a-158b may be sized to engage a compliant or insulative surface of the lead, thereby securing the lead when the first locking member 148 is urged towards the second locking member 150. In other embodiments, the engaging features may engage electrodes of the leads that are to be coupled to a common electrical connection.

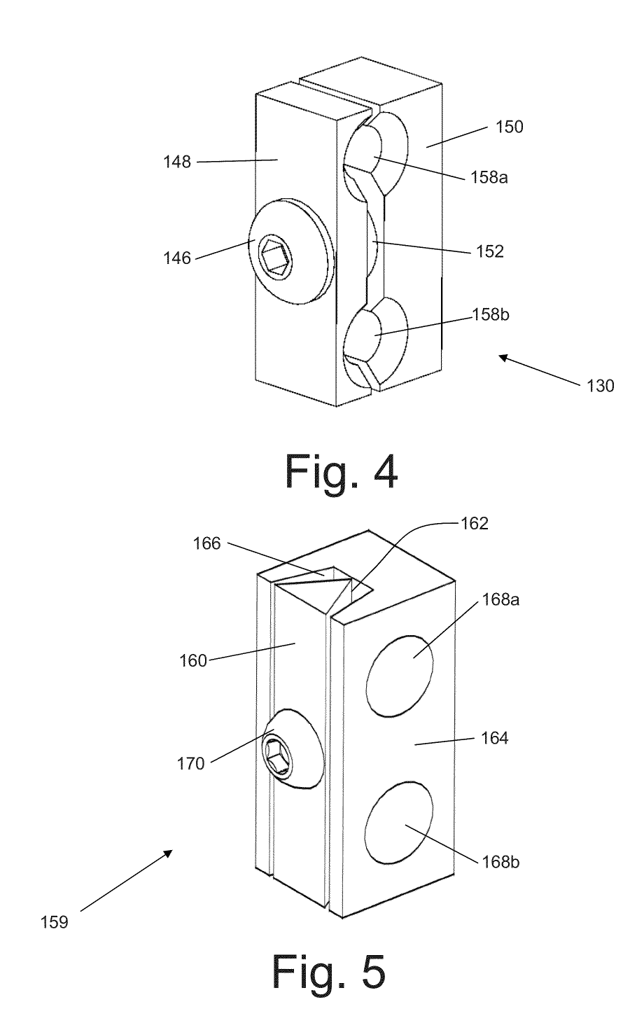

FIG. 5 is an isometric view of a lead locking mechanism 159. In this embodiment, the first member 160 may be a member having a triangular cross-section shape and an engaging edge 162. The second member 164 may have a receiving channel 166 for receiving the first member 160. In certain embodiments, two side bores 168a-168b intersect the receiving channel 166. A coupling member, such as a cap screw 170 couples the first member 160 to the second member 164. In this embodiment, the two side bores 168a-168b are sized to receive the proximal end of the leads (not shown). When the leads are in position within the lead locking mechanism 159 and the cap screw 170 is turned, the engaging edge 162 of the first member 160 is able to exert more engaging pressure onto the lead than would be possible with an engaging surface such as used in the embodiment illustrated with reference to FIG. 4.

As previously discussed, the coupling members 146 of FIG. 4 or 170 of FIG. 5 may be cap screws. In certain embodiments, the cap screws may have torque transferring features such as a socket or slot to couple with a driver. In some embodiments, the cap screws may have generally smooth shafts with threads limited to the distal or insertion end portion of the shaft. In other words, the threads may be formed close to the distal end of the shafts.

As previously described with reference to FIG. 1, the assembled IPG system 100 comprises the header 102 connected to the IPG 104 and stimulation leads 106a and 106b. As is well known in the art, the IPG 104 is capable of being implanted within a body that is to receive electrical stimulation from implantable pulse generator.

In this illustrative example, the stimulation leads 106a and 106b are connected to the implantable pulse generator 104 via the header 102. The leads 106a and 106b may be detached from the pulse generator 108 as desired by applying detaching force and removing the proximal end (not shown) of the leads 106a and 106b from the respective strain relief port 110a-110b. Similarly, the leads 106a and 106b may be inserted into the header 102 by pushing the proximal end into the appropriate strain relief port 110a-110b.

Once the leads are properly inserted, the lead stop member 144 (FIG. 3a) prevents the leads going further, thus assuring proper longitudinal positioning. The lead locking mechanism 130 may then be engage to lock the lead end in position.

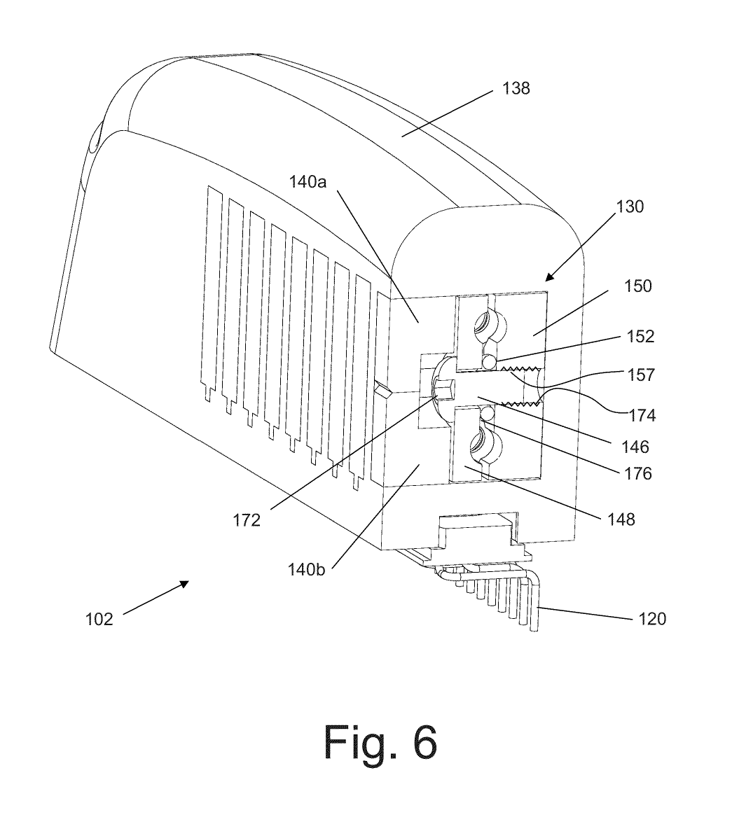

FIG. 6 is a section view cut through the assembled header 102 at the longitudinal location of the locking mechanism 130. As illustrated, the header housing 138 houses the first septum seal member 140a, the second septum seal member 140b, the first locking member 148, the second locking member 150, the compliant member 152, and the cap screw 146. To lock the lead locking mechanism 130, a screw driver or socket driver (not shown) may be inserted between the first septum seal member 140a and the second septum seal member 140b to engage a torque transferring feature defined on the head of the cap screw 146. A clockwise rotation of the cap screw 146 will thus drive the first locking member 148 towards the second locking member 150 and secure the leads positioned between the locking members.

Once the locking mechanism has secured the leads to the header, the connector electrodes 112a-112b (FIG. 1) are in electrical contact with feed-through wires 120. The feed-through wires 120, therefore, connect the connector electrodes to pulse generating circuitry (not shown) within the pulse generator 104 (FIG. 1). The connector electrodes 112a-112b are themselves in electrical contact with the stimulation electrodes at the distal ends of leads 106a and 106b because conductors (not shown) in the leads electrically connect the connector electrodes to the stimulation electrodes.

Thus, the pulse generator 104 may generate and send electrical signals via the leads 106a and 106b to the stimulation electrodes. In use, the stimulation electrodes may be placed at a stimulation site (not shown) within a body that is to receive electrical stimulation from the electrical signals. The stimulation site may be, for example, adjacent to one or more nerves in the central nervous system (e.g., spinal cord). The pulse generator 104 may be capable of controlling the electrical signals by varying signal parameters (e.g., intensity, duration, frequency) in response to control signals. In certain embodiments, the pulse generator 104 may programmed by or be in communication with an external programming device (not shown) which supplies the control signals.

Although certain representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate when reading the present application, other processes, machines, manufacture, compositions of matter, means, methods, or steps, presently existing or later to be developed that perform substantially the same function or achieve substantially the same result as the described embodiments may be utilized. Accordingly, the appended claims are intended to include within their scope such processes, machines, manufacture, compositions of matter, means, methods, or steps.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

1. A medical implantable pulse generator system comprising:
a housing enclosing pulse generating circuitry;
a plurality of feed-through wires coupled to the pulse generating circuitry and extending through the housing;
a first lead having a first plurality of conducting electrodes;
a second lead having a second plurality of conducting electrodes;
a header coupled to the housing, the header comprising:
a first port for receiving a first end of the first lead,
a second port for receiving a second end of the second lead,
a locking mechanism positioned to receive a portion of the first lead and a portion of the second lead, the locking mechanism comprising:
a first locking member adapted to engage a portion of both the first and second leads,
a second locking member adapted to engage a portion of both the first and second leads,
a coupling member coupled to the first locking member and having external threads sized to engage internal threads defined within the second locking member such that a torque force applied to the coupling member is translated into relative lateral movement between the coupling member and the second locking member,
a first plurality of electrical connectors aligned with the locking mechanism and the first port and positioned to engage the first plurality of conducting electrodes
a second plurality of electrical connectors aligned with the locking mechanism and the second port and positioned to engage the second plurality of conducting electrodes, wherein each of the first plurality and the second plurality of electrical connectors comprise at least eight electrical connectors.

2. The implantable pulse generator system of claim 1, further comprising a compliant member positioned between the first locking member and the second locking member.

3. The implantable pulse generator system of claim 1, wherein the compliant member is an O-ring member.

4. The implantable pulse generator system of claim 1, further comprising
a first smooth center bore defined within the first locking member,
a second center bore defined within the second locking member and having the internal threads defined therein.

5. The implantable pulse generator system of claim 4, further comprising
a first engaging face of the first locking member having:
a first semi-circular indent defined within the first engaging face and positioned to engage a portion of the first lead,
a second semi-circular indent defined within the first engaging face and positioned to engage a portion of the second lead,
wherein the first semi-circular indent and the second semi-circular indent are generally perpendicular to the first smooth center bore,
a second engaging face of the second locking member having:
a third semi-circular indent defined within the second engaging face and positioned to engage a portion of the first lead;
a fourth semi-circular indent defined within the second engaging face and positioned to engage a portion of the second lead; and
wherein the third semi-circular indent and the fourth semi-circular indent are generally perpendicular to the second center bore.

6. The implantable pulse generator system of claim 4, further comprising
at least one edge of the first locking member positioned to engage a portion of the first lead,
a channel defined within a side of the second locking member sized to receive the at least one edge of the first locking member,
a first side bore defined within the second locking member and positioned to intersect the channel, wherein the first side bore is sized to receive a portion of the first lead, and
a second side bore defined within the second locking member positioned to intersect the channel, wherein the second side bore is sized to receive a portion of the second lead.

7. The implantable pulse generator system of claim 1, further comprising:
a torque transferring feature defined on a proximal end portion of the coupling member,
a first sealing member positioned proximal to torque transferring feature,
a second sealing member positioned proximal to the torque transferring feature and engaging the first sealing member to form a seal between the first sealing member and the second sealing member.

8. The implantable pulse generator system of claim 1, further comprising:
at least one support having a plurality of wire bores such that the plurality of feed-through wires pass from the interior of the housing through the wire bores and into the header,
a plurality of seals wherein each seal is positioned between each of the feed-through wires and the wire bores,
at least one base coupled to the housing having at least one aperture sized to accommodate the at least one support, and
at least one seal between the at least one aperture and the at least one support.

9. The implantable pulse generator system of claim 1, wherein the electrical connectors are annular electrical connectors comprising a circular support member and a spring member for engaging an electrode of a lead.

10. The implantable pulse generator system of claim 1, further comprising a stop member positioned to stop movement of first the plurality of conducting electrodes relative to the first plurality of electrical connectors during assembly of the system.

11. The implantable pulse generator system of claim 10, wherein the stop member is also positioned to stop the movement of the second plurality of conducting electrodes relative to the second plurality of electrical connectors during assembly of the system.

12. A header component for a medical implantable pulse generator comprising:
a first plurality of spring members adapted to engage a first plurality of electrodes of a first lead and aligned longitudinally to match an electrode spacing of the first lead,
a second plurality of spring members adapted to engage a second plurality of electrodes of a second lead and aligned longitudinally to match the electrode spacing of the second lead,
a first plurality of spring housings adapted to partially house the first plurality of spring members, wherein each spring housing in the first plurality of spring housings is electrically coupled to a feed-through wire,
a second plurality of spring housings adapted to partially house the second plurality of spring members, wherein each spring housing in the second plurality of spring housings is electrically coupled to the feed through wire, wherein each of the first plurality and the second plurality of spring housings comprise at least eight spring housings,
a lead locking mechanism including:
a first locking member positioned to engage a non-electrical conducting surface of the first lead when the first plurality of spring members engage the first plurality of electrodes of the first lead and positioned to engage a non-electrical conducting surface of the second lead when the second plurality of spring members engage the second plurality of electrodes of the second lead,
a second locking member positioned to engage the non-electrical conducting surface of the first lead when the first plurality of spring members engage the first plurality of electrodes of the first lead and positioned to engage the non-electrical conducting surface of the second lead when the second plurality of spring members engage the second plurality of electrodes of the second lead, and
a coupling member coupling the first locking member to the second locking member.

13. The header component of claim 12, further comprising a compliant member positioned between the first locking member and the second locking member.

14. The header component of claim 12, further comprising
a first smooth center bore defined within the first locking member,
a second center bore defined within the second locking member having internal threads defined therein, and
external threads defined on a surface of the coupling member, the external threads sized to engage the internal threads such that a torque force applied to the coupling member is translated into relative lateral movement between the coupling member and the second locking member.

15. The header component of claim 14, further comprising
a first engaging face of the first locking member having:
a first semi-circular indent defined within the first engaging face and positioned to engage a portion of the first lead,
a second semi-circular indent defined within the first engaging face and positioned to engage a portion of the second lead,
wherein the first semi-circular indent and the second semi-circular indent are generally perpendicular to the first smooth center bore,
a second engaging face of the second locking member having:
a third semi-circular indent defined within the second engaging face and positioned to engage a portion of the first lead;
a fourth semi-circular indent defined within the second engaging face and positioned to engage a portion of the second lead; and
wherein the third semi-circular indent and the fourth semi-circular indent are generally perpendicular to the second center bore.

16. The header component of claim 14, further comprising
at least one edge of the first locking member positioned to engage a portion of the first lead and a portion of the second lead,
a channel defined within a side of the second locking member sized to receive the at least one edge of the first locking member,
a first side bore defined within the second locking member and positioned to intersect the channel, wherein the first side bore is sized to receive a portion of the first lead,
a second side bore defined within the second locking member positioned to intersect the channel, wherein the second side bore is sized to receive a portion of the second lead.

17. The header component of claim 12, further comprising:
a torque transferring feature defined on a proximal end portion of the coupling member,
a first sealing member positioned proximal to the torque transferring feature,
a second sealing member positioned proximal to the torque transferring feature and engaging the first sealing member to form a seal between the first sealing member and the second sealing member and providing temporary access to the torque transferring feature.

18.-26. (canceled)
